(19) United States
(12) Patent Application Publication
(10) Pub. No.: US 2008/0317836 A1
(43) Pub. Date: Dec. 25, 2008

(54) Topical compositions for anti-aging skin treatment

(75) Inventors: Peter Ladislaus Dorogi, Easton, PA (US); John Patrick McCook, Frisco, TX (US); David Bruce Vasily, Bethlehem, PA (US)

Correspondence Address: THEODORE JAY;APT 600, 16 N CHATSWORTH AVE, LARCHMONT, NY, 10538, US

(73) Assignee: DISCOVERY PARTNERS LLC

(21) Appl. No.: 11/820,426

(22) Filed: Jun. 19, 2007

(51) Int. Cl. A61K 33/34; A61K 9/127; A61P 17/00

(52) U.S. Cl. 424/450; 424/630; 424/638

(57) ABSTRACT

This invention discloses anti-aging skin treatment compositions and methods that utilize plant-based copper and zinc antioxidant complexes.

### FIELD OF THE INVENTION

This invention describes topical compositions and methods of use for skin treatment formulations containing salts of copper antioxidant pigment complexes enhanced with water soluble zinc salts. This invention relates to therapeutic use of mixtures of salts of plant-based copper antioxidant complexes and zinc for treating, preventing, or reversing signs and symptoms of sun damage, intrinsic aging, such as facial lines and wrinkles, age spots or pigmentary changes, blotchy or uneven skin color, skin laxity and rough or dry skin surfaces.

### BACKGROUND OF THE INVENTION

U.S. application Ser. No. 11/496,155 filed, Jul. 31, 2006, by Dorogi, Vasily and McCook titled “SKIN TREATMENT COMPOSITIONS CONTAINING COPPER-PIGMENT COMPLEXES” describes therapeutic substances and methods for treating, preventing, reversing or inhibiting skin disorders characterized by redness and broken small blood vessels on the face, associated with, but not limited to rosacea, as well as skin disorders with chronic inflammation or sun damage, by topical application of compositions containing copper bound to a botanical pigment and delivered in a suitable pharmaceutical or cosmeceutical vehicle.

Further research by the inventors has shown that mixtures of copper-antioxidant pigment complex and zinc-antioxidant pigment complexes formulated in a topical cosmetic or pharmaceutical vehicle, can be used successfully to reduce or reverse damage to the skin caused by ultraviolet radiation, environmental pollutants, cigarette smoke, intrinsic aging, or skin aging and damage caused by a combination of these factors. The inventors have developed improved skin delivery systems for the mixture of copper-antioxidant pigment complexes and zinc-antioxidant pigment complexes that deliver anti-aging benefits when applied to the skin.

### SUMMARY OF THE INVENTION

In the present invention, cutaneous topical delivery of copper and zinc is accomplished by binding copper and zinc, respectively, with botanical pigments. The copper-pigment complex is used alone or preferably in combination with a zinc-pigment complex.

The term “pigment”, when used in conjunction with copper or zinc, covers botanical or naturally-derived chromophoric molecules, or chemically modified botanical chromophores, possessing antioxidant properties. The copper-pigment complex utilized and demonstrated here, in a non-limiting way, is sodium-copper-chlorophyllin. The copper-pigment complex can also be referred to as a copper-antioxidant pigment complex, a copper-plant complex, or a copper pigment antioxidant complex. The zinc-pigment complex utilized and demonstrated here, in a non-limiting way, is sodium-zinc-chlorophyllin complex. The zinc-pigment complex can also be referred to as a zinc-antioxidant pigment complex, a zinc-plant complex, or a zinc pigment antioxidant complex. The copper-pigment and zinc-pigment complexes are delivered into the skin with a penetration-enhancing vehicle.

In this way, the copper-pigment complex activity is supplemented or enhanced by the addition of a zinc-pigment complex.

To further improve skin penetration, the copper-pigment complex combination with the zinc-pigment complex and its aqueous vehicle can be encapsulated within liposomes. The liposomes used are submicron size, with the majority of the liposomes of diameters below 350 nanometers or 0.35 microns.

This invention delivers copper in conjunction with zinc to binding sites in the skin, where they can be utilized by enzymes and wound-healing copper-peptides; for example, for preventing, reducing, and eliminating the signs and symptoms of photodamage.

This invention also combines copper, in conjunction with zinc, with copper-dependent and zinc-dependent antioxidant enzymes, respectively, which are responsible for the elimination of free radicals generated in the skin by ultraviolet light, reactive oxygen metabolites, and microbe activity.

The disclosed method supports copper-binding enzymes and peptides and zinc-binding enzymes which are active in repair and replacement of damaged skin tissue.

This invention simultaneously supplies to the skin, besides copper and zinc, botanical pigments which are natural antioxidants, so as to further reduce oxidation damage.

These goals are achieved with the use of therapeutic units that consist of a copper ion metallically bound to a suitable botanical pigment and a zinc ion metallically bound to a suitable botanical pigment. Treatment involves cutaneous application of such a copper-pigment complex and zinc-pigment complex delivered in a pharmaceutical or cosmeceutical vehicle that facilitates penetration of the complexes into the skin. In one embodiment of the invention, the copper-pigment complex is sodium-copper-chlorophyllin and the zinc-pigment complex is sodium zinc chlorophyllin. The copper-pigment complex together with the zinc pigment complex, and its carrier or vehicle can be encapsulated in a submicron liposome, with the majority of liposomes having diameters less than 350 nanometers in terms of the average Zeta-potential. These liposomes result in increased skin penetration by the copper-pigment complex and the zinc-pigment complex.

In therapeutic applications the composition containing the copper-pigment complex and zinc-pigment complex can be combined with, or applied in conjunction with, other pharmaceutical, cosmeceutical, or nutritional treatments for treatment of skin hyperpigmentation or age spots, blotchy skin, facial lines and wrinkles, or other manifestations of photodamage or environmental damage to the skin. Topical application of the copper-pigment complex and zinc-pigment complex can be sequential or simultaneous with other topical or even systemic treatments, for example, by injection, inhalation, or ingestion. Simultaneous delivery of the copper pigment complex, the zinc-pigment complex, and another pharmaceutical preparation can, for example, be accomplished by using a co-dispensing applicator.

Combining the compositions and processes utilizing the copper-pigment complex and the zinc-pigment complex with currently established anti-aging technologies bring additional benefits to those technologies. Examples of anti-aging topical skin treatments include combinations of the copper-pigment complex and the zinc-pigment complex with one or more compounds that include peptides, sunscreens, isoflavones, flavenoids, isoprenoids, quinones, metalloprotease enzyme inhibitors, or other recognized anti-aging actives. Co-dispensing the copper-pigment complex and zinc-pigment complex with other actives formulated at an acidic pH will enhance the release of ionic copper and zinc from pigment complexes. Desired kinetics of copper and zinc ion release from such preparations can be modulated by proper setting of the pH during formulation.

### DETAILED DESCRIPTION OF THE INVENTION

Damage to connective tissue proteins, namely collagen and elastin, reduces the tensile strength of the skin. Structural weakening of the connective tissue is a probable cause of telangiectasias following distension and thinning of blood vessel walls. Increased tissue blood volume is visible through the skin surface and is responsible for the appearance of diffuse redness.

Increased cross-linking of connective tissue proteins due to increased bioavailability of copper, enhanced with zinc, can

1) improve tensile strength and elasticity of skin and thereby diminish the appearance of fine lines and wrinkles 2) reduce the appearance of blotchy skin and uneven coloration, 3) improve skin radiance, and reduce other signs and symptoms of photodamage or environmental damage to the skin.

Binding copper with an antioxidant botanical pigment imparts important additional protection against oxidative damage in the skin, beyond that naturally present for repair of damaged cells and macromolecules. Physiological wound healing includes the body's own production and secretion of superoxide radicals (.O₂⁻) by phagocytic cells, in order to destroy invading microorganisms. Although the superoxide radical is itself not very damaging, it can react with transition metals such as ferrous iron (Fe⁺²) and cuprous copper (Cu⁺²) to generate the extremely reactive hydroxyl radical .OH⁻. To offset such potentially harmful side-effects, copper-delivery compositions are employed wherein copper is applied in forms that provide their own antioxidant protection; namely, copper bound with protective antioxidant botanical pigments.

Elimination of the superoxides from the skin is largely done by superoxide dismutases (SODs). The predominant form of SOD in the skin is the Cu⁺²/Zn⁺²⁻ containing dimeric form of the enzyme. It is disclosed here that increasing the amount of copper, or copper and zinc, in the skin can result in increased SOD activity, thereby reducing free radical activity and associated skin inflammation.

In one embodiment of the invention, zinc, in the form of sodium zinc chlorophyllin is used to supplement the copper-pigment complex. The role of zinc in the mediation of collagen destruction is well known. The destruction and removal of damaged connective tissue is a critical phase of skin re-modeling after wounding or pathological damage. The early destructive phase of collagen re-modeling is carried out by the enzyme collagenase, which is a metalloenzyme containing extrinsic (surface-bound) cations, mostly Ca²⁺, that stabilizes the tertiary conformation of the enzyme, and an intrinsic transition metal ion, Zn²⁺, must be present at the catalytic site.

Collagenase acts by splicing through all three collagen fibrils comprising the helical structure of collagen. Following any type of significant dermal damage, collagenase is secreted by dermal fibroblasts and is the main protease for breaking down damaged collagen. These events create small peptides that contain high-affinity copper binding sites, most notably the wound-healing peptide known as GHK. Topical treatment with zinc preparations speeds up skin healing, suggesting in turn that the increased concentration of zinc increases collagenase concentration.

Zinc has been reported to facilitate the early phase of connective tissue re-modeling in another way; namely, by slowing down the secretion of new collagen from fibroblasts. The advantage is reduced scarring. The rate-limiting reaction step before newly synthesized collagen is released from fibroblasts is the creation of hydroxyproline structures in the collagen, since these structures are essential for its helical form. It has been reported that Zn²⁺ inhibits prolyl hydroxylation at its typical physiological concentrations.

In summary, zinc must be available for skin remodeling to be initiated by digestion of damaged collagen and for the generation of copper peptides such as GHK that influence subsequent healing steps. Zn²⁺ and Cu²⁺ work synergistically in the healing of connective tissue. Zinc has been reported to be present in the skin at a twenty-fold higher concentration than copper.

Binding zinc with an antioxidant botanical pigment imparts important additional protection against oxidative damage while delivering exogenous zinc ions to supplement the collagen remodeling process described above. In this invention, zinc, in the form of sodium zinc chlorophyllin complex, is used in a topical anti-aging skin treatment together with a copper-antioxidant pigment, namely, sodium copper chlorophyllin complex.

Other soluble zinc salts can enhance the activity of copper as described above and can be used in place of the sodium zinc chlorophyllin to supplement the copper-pigment complex. However, the zinc-antioxidant plant pigment complex is preferred. Examples of suitable water soluble zinc salts would include zinc phosphate, zinc chloride, zinc sulfate, zinc PCA, zinc gluconate, zinc glycinate, zinc lactate and zinc acetate.

Zinc-antioxidant pigment complex, such as sodium zinc chlorophyllin, is used in combination with a copper-pigment complex synergistically to heal connective tissue associated with sun damage.

Formulations of copper-pigment complex in combination with zinc-pigment complex or soluble zinc salts as described above, must be formulated at a pH of 7.0 or above to maintain stability of the complexed copper-pigment and to avoid conversion of the complexed copper to the reactive Cu⁺² form prior to application to the skin.

The combination of a copper-botanical-antioxidant complex and a zinc-botanical-antioxidant complex disclosed herein to provide increased safety and effectiveness over use of copper-botanical-antioxidant complexes alone. The reason is that zinc provides the intrinsic benefits already listed above, Zinc also competes with copper at low-affinity copper binding sites, thereby reducing the risk of copper toxicity, a condition associated with poor liver function and failure to clear excess free copper from the body, as in Wilson's Disease, The combination of copper and zinc, in the form of botanical-antioxidant complexes both with enhanced delivery in liposomal dispersions, is a meaningful advance. The copper-antioxidant pigment complex, in combination with zinc-antioxidant pigment complex, is applied to the skin in a pharmaceutical or cosmeceutical vehicle, containing one or more penetration-enhancing ingredients. Suitable penetration enhancers include, but are not limited to, ethoxydiglycol (also known as diethylene glycol monoethyl ether, commercially available as TRANSCUTOL from Gattefosse', Inc. and TRIVALIN CG from Tri-K Industries), 1,3-butylene glycol, isopentyl diol or isoprene glycol, dimethyl isosorbide, propylene glycol, 1,2-pentane diol, propylene glycol, 2-methyl propan-2-ol, propan-2-ol, ethyl-2-hydroxypropanoate, hexan-2,5-diol, di-(2-hydroxypropyl) ether, pentan-2,4-diol, acetone, polyoxyethylene (2) methyl ether, 2-hydroxypropionic acid, 2-hydroxyoctanoic acid, propan-1-ol, 1,4 dioxane, tetrahydrofuran, butan-1,4-diol, propylene glycol dipelargonate, polyoxypropylene 15 stearyl ether, octyl alcohol, polyoxyethylene ester of oleyl alcohol, oleyl alcohol, lauryl alcohol, dioctyl adipate, dicapryl adipate, diisopropyl adipate, diisopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hyroxyoctanoic acid, dimethyl sulphoxide, methyl sulfonyl methane (MSM), n,n-dimethyl acetamide, n,n-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, 1-dodecylazacyloheptan-2-one, acetamide MEA, tetrahydropiperine, various PEG glyceryl ethers, Levomenol [(−)-6-Methyl-2-((4-methyl-3-cyclohexen-1-yl)-5-hepten-2-ol], combinations thereof, and the like.

The amount of penetration enhancer in the compositions of the present disclosure can be less than about 20% by weight of the composition, in embodiments from about 0.1% by weight to about 10% by weight of the composition. In some embodiments, it is desirable to utilize the penetration enhancer in an amount of about 5% by weight of the composition. Preferred penetration enhancing ingredients include the diglycols; pentylene glycol, isopentyl glycol,1, 3-butylene glycol, 1,4-butylene glycol and ethoxydiglycol, or mixtures of these diglycols.

The copper-antioxidant and zinc-antioxidant pigment complexes and an aqueous vehicle can be delivered within a liposomal dispersion, wherein the lipid shell of the liposome consists of a phospholipid, Suitable phospholipids and/or phospholipid derivatives include, but are not limited to, lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, combinations thereof, and the like.

In some embodiments, a lecithin derived from egg or soybean can be utilized as the phospholipid. Such lecithins include those commercially available as PHOSPHOLIPON® 85G, PHOSPHOLIPON® 90G, and PHOSPHOLIPON® 90H (the fully hydrogenated version of PHOSPHOLIPON® 90G) from American Lecithin Company, Oxford, Conn. Other suitable lecithins include LECINOL S-10® lecithin from Nikko Chemicals. Preferably, a vegetable-based phosphatidyl choline or soy lecithin is used to make the liposomes. Liposomes of this type were made in situ in a suitable cosmetic or pharmaceutical vehicle by separately dissolving the copper-pigment complex and the zinc-pigment complex in water and encapsulating the aqueous copper-antioxidant complex as well as the aqueous zinc-antioxidant complex with Phospholipon 85G®. Lecithin of this type was obtained from the American Lecithin Company, originally manufactured by Phospholipid GmbH (Cologne, Germany) and typically contains more than 50% linoleic acid. Alternatively, the liposomes can be made using diacylglycerol-polyethyleneglycol (DAG-PEG) lipids. In one embodiment of the invention, liposomes containing sodium copper chlorophyllin are made using PEG-12 glyceryldimyristate supplied by BioZone Laboratories, Inc. of Pittsburgh, Calif.

The invention combines four types of ingredients: 1) copper and zinc ions, bound to 2) antioxidant botanical pigments, 3) skin penetration enhancing vehicles containing certain diglycols, and 4) lipids in the form of liposomes.

Another aspect of the invention is a method for controlled release of copper and zinc ions in the skin, based on suitable formulation of the pH of the therapeutic unit compared with the internal pH of the skin. In the case of sodium-copper-chlorophyllin and sodium-zinc-chlorophyllin, copper and zinc are bound to the chlorophyllin by a metallic bond and can be replaced by two protons: a more acidic environment releases the copper as well as the zinc ions, increasing the concentration of the free form, Cu⁺² and Zn⁺². It is thereby another aspect of the invention that the therapeutic unit is formulated in a vehicle at a slightly alkaline pH: typically, a pH range of 7.2 to 7.6 gives good results.

Aqueous solutions of sodium-copper-chlorophyllin complex as well as aqueous solutions of sodium-zinc-chlorophyllin complex are typically alkaline, in the pH-range 8.0-9.5 prior to any pH adjustment via acidifying agents or buffering agents. When liposomal dispersants are used to enhance skin penetration, the pH of the liposomal dispersion is adjusted to a slightly alkaline pH of 7.2-7.6 to stabilize sodium-copper-chlorophyllin.

The more acidic environment of the skin's outer mantle would normally enhance release of copper and zinc ions bound to the antioxidant pigment. Encapsulation by liposomes acts as a shield against this “acid shock”, resulting in deeper skin penetration, slower release, and potentially enhanced biological utilization of copper and zinc. In a “proof-of-principle” study with one human subject, increased utilization of copper was demonstrated for the liposomal system; the experiment is described in detail as Study 1 below. This proof-of-principle study indicates that 1) the aqueous composition containing sodium-copper-chlorophyllin and diglycol penetration-enhancing agents delivers copper to the enzyme tyrosine hydroxylase situated at the base of the epidermis and 2) encapsulation of the copper-pigment complex within a liposome increases the amount of copper delivered. The following studies were conducted to evaluate the effectiveness of such compositions.

A small study, on one subject, was conducted to see whether encapsulation of the sodium-copper-chlorophyllin within the liposome enhanced skin tanning, i.e., increased penetration and bioavailability of copper to the melanin-producing enzyme, tyrosine hydroxylase. The study compared treatment with 0.10% by weight sodium-copper-chlorophyllin in a treatment gel composition as described in Exhibit 2, with and without liposome encapsulation. Three areas on the back received 1.0 MED simulated solar radiation: one area had received no treatment, one area had been pre-treated with the gel without liposome and the third area was treated with the liposome gel. An expert blinded grader scored the resulting skin tanning at 10 days after irradiation, using the Degree of Tanning scale 0 to 10 (darkest). The control, untreated area showed very little tanning and was given a tanning score of 1-2; the area treated using the gel without liposome was scored as 6.0-6.5; the area treated with the liposome gel was scored as 8. These results indicate copper from the copper-pigment complex applied in a vehicle with penetration-enhancing agents reaches and is picked up by melanocytes, with and without the liposome, but that the liposome enhances penetration beyond that achievable with the penetration enhancers used. Controlled dissociation of the sodium-copper-chlorophyllin complex is possible, in part because of the high stability of this complex. Normally, inorganic and organic copper salts, including copper-peptides, are not very stable with regard to binding copper. The inherent stability and lack of reactivity of sodium-copper-chlorophyllin is essential for this process to work successfully. The stability of this copper-pigment complex is displayed by exposing a sample of the material to intense pulsed light (IPL) flashes in the visible spectral range absorbed by this dye. Stability was monitored by measurement of the dye's full absorbance spectrum, since degradation of the dye would be detectable as changes in the absorbance spectrum. Even after application of ten pulses at the maximum fluence of the flash lamp, the absorbance spectrum of sodium-copper-chlorphyllin remained unchanged, demonstrating high thermal and light stability for this material.

In nature, many types of botanical pigments protect plants against the free radicals generated from molecular oxygen. Antioxidant pigments are essential for the survival of plants, and the pigments usually contain a metal-ion such as magnesium, zinc, or copper. Although our experiments utilized sodium-copper-chlorophyllin and sodium-zinc-chlorophyllin, topical application of other examples of botanical pigments containing transition metals and possessing free radical scavenging properties are considered to be part of this invention. A non-limiting list of examples of such pigments includes carotenoids, chlorophylls, anthocyanins, betalains and phycobilins.

Bound Cu⁺² ions and Zn⁺² ions are loaded into the liposomes as an aqueous solution of sodium-copper-chlorophyllin and sodium-zinc-chlorophyllin. Chlorophyllin itself is a highly effective antioxidant and is expected to neutralize any free radical load put on the skin by reactions between Cu⁺², hydrogen peroxide, and ultraviolet light or combinations of these.

In one preferred embodiment of this invention, the carrier vehicle of the copper-pigment complex as well as the zinc-pigment complex is a liposomal dispersion, containing submicron diameter lecithin liposomes, with the majority of the liposome diameters falling below 350 nm or 0.35 micron. Typically, the average particle diameter is between 150-350 nanometers (0.15-0.35 micron). The average liposome particle size can be measured via laser diffraction using a Malvern ZetaSizer (MalvernInstruments, Ltd., Worcestershire, UK) or similar equipment suitable for measuring dispersions of submicron particles.

Copper and zinc ions are favored to dissociate from chlorophyllin due to the acidity of the skin compared with the formulated higher pH of the composition. This release process is expectedly slowed by liposome encapsulation. Alternatively, dissociation of the copper-pigment complex and the zinc-pigment complex can be increased by increasing the skin temperature; for example, by optical or sonic heating of the skin; or, the release can be slowed by cooling the skin.

Because copper and zinc ions bind with many skin peptides and proteins, benefits of this type of topical treatment depend on the applied copper and zinc reaching the desired copper-binding and zinc-binding sites; namely, dependent enzymes and peptides. The combination of these particular liposomes, characterized by their high stability and small size, loaded with copper and zinc bound to a suitable antioxidant pigment, delivered within a penetration-enhancing vehicle, are all essential elements of this invention.

The present invention provides materials and compositions that enhance cutaneous physiological functions dependent on the presence of copper and/or zinc. These functions include repair and growth of connective tissue, regulation of mitochondrial energy metabolism, formation of melanin, and reduction of free radicals.

The invention discloses methodology consisting of:

1) a composition containing a non-acidic formulation of copper and zinc, each bound to a botanical pigment possessing antioxidant qualities, the composition thus containing a copper-botanical-antioxidant complex and a zinc-botanical-antioxidant complex;
2) identification of a carrier vehicle containing penetration enhancing agents and encapsulation of the copper-antioxidant-pigment complex and zinc-antioxidant-pigment complex in stable, submicron diameter liposomes;
3) identification of physiological copper and zinc binding sites in the skin that modulate intrinsic aging, or aging due to sun exposure or environmental factors, the signs including pigmentary changes, blotchy or uneven patches, sallowness or lack of radiance, lines and wrinkles, laxity, and surface roughness;
4) topical application to skin of the encapsulated copper-antioxidant-pigment complex and zinc-antioxidant-pigment complex composition resulting in enhanced penetration of the composition to cutaneous copper and zinc binding sites; and,
5) simultaneous delivery of skin pH-activated copper and zinc ions and plant-based antioxidant pigments to treat said skin disorders and to protect against further skin damage.

Copper ion (Cu⁺²) that is metallically bound to a botanical pigment, chlorophyllin, can function as a biologically effective copper-delivery system when suitably formulated for topical application. The pigment part of the complex can function as an antioxidant in the skin, thereby reducing skin inflammation. The list of copper-dependent enzymes that are normally active in the skin, and thereby affected, includes lysyl oxidase (cross-links collagen fibrils to form the structurally supportive collagen fibers), superoxide dismutase (active in the reduction of damaging oxygen species), tyrosine hydroxylase (important in the synthesis of melanin and the neurotransmitter norepinephrine). Other copper-containing enzymes found in the skin are cytochrome C oxidase (involved in production of the skin's high-energy metabolic substrates) and dopamine beta-hydroxylase (involved in regulation of dopamine and norepinephrine). By donating copper to enzymes and copper-dependent wound-healing peptides, via the compositions and processes disclosed herein, treat all copper-dependent needs of the skin.

The importance of copper ions in skin biology has been explained given the necessary presence of copper at catalytic sites of various enzymes, noting also its association with small peptides, such as GHK, which modulate recovery from injury. Whereas botanical pigments such as chlorophylls and carotenoids have been used previously in skin care formulations for their antioxidant potential, binding copper ions with such botanical pigments, specifically to deliver copper, is new.

The role of zinc in skin remodeling and the generation of copper peptides that influence subsequent redevelopment steps in building new collagen has been previously described. Zn²⁺ and Cu²⁺ can work synergistically towards the healing of connective tissue. The use of zinc bound to an antioxidant pigment, in combination with copper-antioxidant pigment, is an important element of this invention.

Chlorophyll and chlorophyllin have been used to sanitize and deodorize wounds and skin, to treat burns, blisters, ulcerations, psoriasis, inflammatory skin conditions and as additives to hair growth preparations. Copper ions, in bound forms such as copper sulfate, copper glycosides, copper sucralphate and copper gluconate, have had prior use as topical anti-inflammatory agents and in the treatment of spider veins, cellulite, poison ivy, as well as in antiviral compositions. Copper peptides have been described in the patent literature for a variety of skin treatments but copper peptides generally lack the intrinsic stability and antioxidant protection required for controlled dermal release of copper ions and which is afforded by compounds of copper complexed to plant antioxidant pigments.

Zinc salts in the form of zinc chloride, zinc PCA, zinc citrate, zinc undecylenate, for example, have been used topically as antimicrobials, astringents and humectants. There is currently no recognized pharmaceutical of dermatological use for salts of zinc-antioxidant pigment complexes and specifically, sodium-zinc-chlorophyllin.

Sodium-copper-chlorophyllin has been previously used as a photomodulation agent in the skin, whereby light energy absorbed by this compound, for example from a laser, is transferred to a neighboring, endogenous skin-cell pigment, thereupon “energizing” the cell. The copper-pigment can thereby function as a skin or hair growth stimulation agent. However, copper of this form has not been viewed to have an active role, other than stabilizing the molecular structure of chlorophyllin.

Copper-chlorophyllin has been used previously as an internal deodorant in tablet form, in combination with proteolytic enzymes for the debridement and healing of ulcerative wounds and as a colorant in dentifrice, bone cement, and certain dry foods. Oil-soluble copper-chlorophyll and water soluble sodium-copper-chlorophyllin have not been used commercially in pharmaceutical or cosmeceutical skin care products, except for limited use as a deodorant and wound healing additive to products used to treat deep, open wounds such as decubitus ulcers and colostomy openings. However, neither copper-chlorophyll nor sodium-copper-chlorophyllin have ever been used or suggested for use in the topical treatment of aged or photoaged skin.

It can be assumed that sodium-copper-chlorophyllin and sodium-zinc-chlorophyllin have not been used in topical pharmaceutical, dermatological or cosmetic products in any significant way simply because these materials are dark-green pigments, even at low concentrations. For example, both sodium-copper-chlorophyllin and sodium-zinc-chlorophyllin exhibit a dark-green color in water at concentrations as low as 0.05% by weight. Topical use products are typically uncolored or lightly colored with dyes to avoid staining of the skin or with insoluble and non-staining inorganic pigments. Green is, of course, not a natural skin tone.

A total concentration of sodium-copper-chlorophyllin and sodium-zinc-chlorophyllin of up to 0.1% by weight for very light skin and up to 0.5% by weight for very dark skin can be used topically; that is, the liposomal dispersion of the aqueous solution of the sodium-copper-chlorophyllin and sodium-zinc-chlorophyllin complexed is absorbed and is not visibly evident after application to the treatment area. This is particularly important since the physical location of the treatment areas will typically include application to the face. The ratio of sodium-copper-chlorophyllin to sodium-zinc-chlorophyllin used in the invention can range from 50:1 to 1:50 with a total applied concentration of sodium-copper-chlorophyllin and sodium-zinc-chlorophyllin that ranges from 0.005% to 5.0% by weight and a preferred total concentration that ranges from 0.025% to 0.1% by weight.
1. Based on history of use, one would not expect a priori that aqueous solutions or dispersions of sodium-copper-chlorophyllin and sodium-zinc-chlorophyllin would readily penetrate intact skin.
2. Based on history of use, one would not expect a low level of copper-chlorophyllin, for example, 0.1% or less by weight, a level used as a colorant in dentifrice and foods, to produce visible improvements in skin condition;
3. Based on history of use, one would not expect visible reductions in pore size and uneven skin coloring, increased tensile strength of skin, reduction in facial and lip lines and wrinkles, reduction in under-eye puffiness and dark circles, and improvement in skin smoothness and radiance after twice daily topical use of sodium-copper-chlorophyllin and sodium-zinc-chlorophyllin for 8 weeks;

The potential of sodium-copper-chlorophyllin as a copper-delivery agent, transferring chlorophyllin-bound copper to copper-dependent enzymes in the skin, is new, as is the concept of using the pigment to reduce free radicals produced by the additional free copper load. Additionally, the combination sodium-copper-chlorophyllin and sodium-zinc-chlorophyllin for topical treatment is novel.

The preparation of one such “therapeutic unit”, in an embodiment of the invention that utilizes sodium-copper-chlorophyllin and sodium-zinc-chlorophyllin as the copper and zinc pigment complexes. Botanical pigments invariably possess a metal-ligand binding site: in natural chlorophyll this binding site is occupied by a magnesium atom. The water-insoluble chlorophyll is converted to the very water-soluble form, chlorophyllin, by alkaline hydrolysis that replaces chlorophyll's methyl and phytyl ester groups with a sodium ion. Copper or zinc is substituted for magnesium by treating the chlorophyll with an acid, thereby replacing the magnesium with two hydrogen ions. The protons are replaced by a cuprous copper ion (Cu⁺²) or zinc ion (Zn⁺²) by alkaline hydrolysis with a copper or zinc salt solution.

Liposomes containing sodium-copper-chlorophyllin as well as sodium-zinc-chlorophyllin were prepared by using high linoleic acid lecithin (Phospholipon 85G®) supplied by American Lecithin Company (Oxford, Conn.) or by Phospholipid GmbH (Cologne, Germany). Typical liposome formulations made using lecithin and sodium copper chlorophyllin are detailed below in Exhibit 1 (Formula number JPM-01-003-B) and typical liposome formulations made using lecithin and sodium-zinc-chlorophyllin are detailed below in Exhibit 2 (Formula number JPM-01-003-A):

### Exhibit 1

### Sodium Copper Chlorophyllin Liposome Concentrate Formula # JPM-01-003-B

| Ingredient | % w/w |
|---|---|
| Phospholipon 85G (Phospholipid GmbH) | 10.00 |
| Sodium Copper Chlorophyllin, USP* | 5.00 |
| Hydrolite-5 (Symrise, Inc.) | 3.00 |
| Butylene Glycol | 4.00 |
| Phenoxyethanol | 0.30 |
| Deionized Water | 77.70 |

*Supplied by Seltzer Chemical Inc., Carlsbad, CA

The above liposome formula (JPM-01-03-B) is made by first combining and dissolving the butylene glycol, Hydrolite-5 (1,2-pentane diol), and phenoxyethanol in the deionized water. This mixture is heated to approximately 50° C. before adding the sodium copper chlorophyllin. This mixture is then mixed until the chlorophyllin salt is fully dissolved. The mixture is then cooled to 25-30° C. and the Phospholipon lecithin is added. The aqueous solution of sodium copper chlorophyllin and the lecithin are homogenized with a Waring® type blender, Osterizer®, Eppenbach® or Silversong® homogenizer or a similar mixer capable of high shear mixing. High shear mixing is continued at approximately 7000 rpm for approximately 15 minutes to create a uniform liposome dispersion with the average liposome particle measuring between 150-350 nanometers with the majority of the particle diameters falling below 300 nm. The resulting liposomal dispersion of sodium copper chlorophyllin is a dark green, syrup-like liquid with a pH of 8.5-9.5. Buffer solution can be added to maintain the pH of the final liposome dispersion in the range of 7.5-8.5.
Liposome dispersions of the sodium copper chlorophyllin can also be made by changing the ratio of Phospholipon from the 2:1 ratio of lecithin: sodium copper chlorophyllin used in Exhibit 1 to higher or lower ratios of Phospholipon to chlorophyllin salt.

### Exhibit 2

### Sodium Zinc Chlorophyllin Liposome Concentrate Formula No. JPM-01-003-A

| Ingredient | % w/w |
|---|---|
| Phospholipon 85G (Phospholipid GmbH) | 10.00 |
| Sodium Zinc Chlorophyllin* | 5.00 |
| Hydrolite-5 (Symrise, Inc.) | 3.00 |
| Butylene Glycol | 4.00 |
| Phenoxyethanol | 0.30 |
| Deionized Water | 77.70 |

*Supplied by Franken Biochem Ltd. (Shangdong, Qingdoa, China)

The above liposome formula (JPM-01-003-A) is made by first combining and dissolving the butylene glycol, Hydrolite-5 (1,2-pentane diol), and phenoxyethanol in the deionized water. This mixture is heated to approximately 50° C. before adding the sodium zinc chlorophyllin. This mixture is then mixed until the chlorophyllin salt is fully dissolved. The mixture is then cooled to 25-30° C. and the Phospholipon lecithin is added. The aqueous solution of sodium copper chlorophyllin and the lecithin are homogenized with a Silverson® L4RT homogenizer or a similar mixer capable of high shear mixing.
High shear mixing is continued at 7000 rpm for 15-20 minutes to create a uniform liposome dispersion with the average liposome particle diameter measuring between 150-350 nanometers with the majority of the particles falling below 300 nm. The resulting liposomal dispersion of sodium zinc chlorophyllin is a dark green, syrup-like liquid with a pH of between 9.0-9.5. Buffer solution can be added to maintain the pH of the final liposome dispersion in the range of 7.5-8.5.
Liposome dispersions of the sodium zinc chlorophyllin can also be made by changing the ratio of Phospholipon from the 2:1 ratio of lecithin: sodium zinc chlorophyllin used in Exhibit 2 to higher or lower ratios of Phospholipon to chlorophyllin salt.

In another embodiment of the invention, the liposomal dispersions referenced above as Formula # JPM-01-003-B (Exhibit 1) and Formula # JPM-01-003-A (Exhibit 2) containing sodium-copper-chlorophyllin and sodium-zinc-chlorophyllin, respectively, were used to formulate a topical treatment gel of the following composition shown below in Exhibit 3 (all components are listed in percentage by weight):

### Exhibit 3

### Chlorophyllin Anti-aging Facial Treatment Gel Formula No. JPM-01-019

| Ingredient | % (w/w) |
|---|---|
| Carbopol 940; 2% dispersion | 60.00 |
| 1,3-Butylene Glycol | 4.00 |
| Sodium Lactate, 60% (Ritalac NAL; RITA) | 1.60 |
| Pentylene Glycol (Hydrolite-5) | 4.00 |
| Phenoxyethanol | 0.60 |
| Sodium Hydroxide solution, 25% | 2.40 |
| Sod. Copper Chlorophyllin liposome # JPM-01-003-B* | 0.66 |
| Sod. Zinc Chlorophyllin liposome # JPM-01-003-A** | 0.66 |
| Deionized Water (add a sufficient amount to make) | 100.00 |

*Liposome contains 5% w/w sodium copper chlorophyllin; final concentration of sodium copper concentration in the above anti-aging gel is 0.033% w/w.
**Liposome contains 5% w/w sodium zinc chlorophyllin; final concentration of sodium zinc chlorophyllin in the above anti-aging gel is 0.033%

The final concentration of sodium-copper-chlorophyllin in the above treatment gel (Exhibit 5) is 0.033% w/w. Sodium zinc chlorophyllin, at a concentration of 0.033% by weight was added to the formulation to enhance the efficacy of the copper-antioxidant pigment complex. The pH of the gel formula was typically adjusted to between 7.2-7.6 with NaOH solution. The formula shown in Exhibit 3 above was made by first creating a Carbopol® (Noveon Corporation) gel adjusted to a pH of between 7.2-7.6 with sodium hydroxide solution, adding all ingredients other than the liposome dispersion containing sodium copper chlorophyllin and the liposome containing sodium zinc chlorophyllin. As a last step, the liposome dispersions are mixed uniformly throughout the neutralized gel.
The copper-dependent biological functions have been shown to include, but not limited to, repair and growth of connective tissue, regulation of mitochondrial energy metabolism, formation of melanin, and reduction of active oxygen species. Insomuch as these biological functions of copper are associated with processes for dermal repair and maintenance of dermal health, the biological activity of copper can be enhanced through the addition of Zn⁺² ions to facilitate the collagen remodeling process as previously described. Non-limiting examples of soluble zinc salts that can be used in combination with complexes of copper-antioxidant plant pigment include zinc gluconate, zinc chloride, zinc sulfate, zinc PCA, zinc acetate, inc citrate, zinc lactate, zinc phosphate, zinc pyrophosphate, ammonium zinc tartrate, zinc aspartate, zinc diacetate, zinc dichloride, zinc glutamate, zinc glycinate, and sodium zinc chlorophyllin. The soluble zinc salts can be used together with the copper-pigment complex in equal amounts by weight or in ratios of 1:50 to 50:1 of complexed copper to soluble zinc ions and preferably ratios of 1:20 to 20:1 of complexed copper to soluble zinc ions. In one embodiment of the invention, liposomes of a soluble zinc salt were formulated to enhance the penetration of the zinc ions.
In a further embodiment of the invention, liposomes of both sodium copper chlorophyllin and sodium zinc chlorophyllin are formulated, together with other pharmaceutical or cosmetic ingredients to create an anti-aging gel for topical application as shown in Exhibit 3. The copper antioxidant pigment complex used is sodium copper chlorophyllin but other complexes of copper and plant antioxidant pigments can be used. The concentration of the copper antioxidant pigment can be from 0.0001% and 5.0% by weight but preferably between 0.0001% and 0.5% by weight and specifically in the range of 0.001% and 0.1% by weight. The copper antioxidant pigment complex can be encapsulated and can be enhanced with the addition of water-soluble zinc salts to generate Zn⁺² ions. The concentration of soluble zinc salts can range from 0.0001% to 50% by weight but preferably between 0.0001% and 5.0% and specifically in the range of 0.001% and 1.0% by weight. The ratio of copper antioxidant pigment complex to soluble zinc salt can be from 1:50 to 50:1 and preferably from 1:20 to 20:1 and specifically from 1:5 to 5:1. The copper antioxidant pigment can be formulated into a cosmetically or pharmaceutically acceptable vehicle known to those skilled in the art. Non-limiting examples of pharmaceutical or cosmetic vehicles for topical application to the skin can take the form of a gel, cream, lotion, serum, liquid, semi-solid or solid dispersion, aerosol spray, foam, stick, pencil-type applicator, powder, patch or other solid multi-layered or single layer solid substrate. The cosmetic or pharmaceutical vehicle used for delivery of the copper-antioxidant pigment complex can be formulated with water and liquid, semi-solid or solid emollients, other solvents, humectants, penetration enhancing agents, thickeners, emulsifiers, surfactants, preservatives, buffers or other agents to adjust pH, propellants, certified cosmetic or pharmaceutical colorants or dyes and inorganic cosmetic pigments. The topical composition of the present invention can also contain other various known and conventional cosmetic or pharmaceutical additives known to those skilled in the art of formulating vehicles for delivery of pharmaceutical or cosmeceutical active ingredients.

The anti-aging treatment gel formulation shown in Exhibit 3 was evaluated in an 8 week clinical trial to assess the safety and anti-aging effectiveness of this treatment gel containing a copper-antioxidant complex and a zinc-antioxidant complex on female subjects with photodamaged skin using clinical grading and self-assessment questionnaires.

The general procedures for the clinical study involved the assessment of signs and symptoms of photodamage at baseline and after twice daily treatment for 8 weeks. The signs and symptoms of photodamage included overall (global) photodamage, fine lines and wrinkles, pore size, age spots (solar lentigenes), dyschromia, visual and tactile roughness, radiance, dryness, and skin firmness or laxity (via pinch recoil). A summary of the clinical assessment of the various clinical grading parameters are shown in Exhibit 4 below. All parameters showed a statistically significant improvement after twice daily treatment for 8 weeks with anti-aging treatment gel JPM-01-019.

### Exhibit 4

### Facial Anti-Aging Study

### Mean Values & Statistical Annalysis for Clinical Grading Parameters and Pinch Recoil Measurements (n=10)

| | Baseline | Week 8 |
|---|---|---|
| | (Visit 1) | (Visit 2) |
| GLOBAL ASSESSMENT | | |
| Overall Photodamaged Skin | 6.15 | 5.20 [symbol] (−15.5%) |
| Appearance | | |
| Fine Lines in the Periocular Area | 5.50 | 4.20 [symbol] (−23.5%) |
| Coarse Wrinkles in the Periocular | 4.20 | 3.40 [symbol] (−20.3%) |
| Area | | |
| Visual Roughness | 5.20 | 3.35 [symbol] (−36.7%) |
| Tactile Roughness | 3.60 | 0.70 [symbol] (−83.3%) |
| Skin Radiance | 3.90 | 5.15 [symbol] (38.2%) |
| Pore Size | 4.95 | 3.65 [symbol] (−25.3%) |
| Dyschromia | 5.95 | 4.95 [symbol] (−17.7%) |
| SOLAR LENTIGENES | | |
| Darkness | 2.80 | 2.23 [symbol] (−20.1%) |
| Relative Size | 4.00 | 2.80 [symbol] (−30.0%) |
| IRRITATION/SAFETY | | |
| Erythema | 0.20 | 0.05 (−5.0%) |
| Edema | 0.00 | 0.00 (0.0%) |
| Scaling | 0.00 | 0.00 (0.0%) |
| Dryness | 0.55 | 0.00 [symbol] (−18.3%) |
| Burning | 0.00 | 0.00 (0.0%) |
| Stinging | 0.00 | 0.00 (0.0%) |
| Itching | 0.00 | 0.00 (0.0%) |
| Tightness | 0.25 | 0.00 (−8.3%) |
| Tingling | 0.00 | 0.00 (0.0%) |
| PINCH RECOIL MEASUREMENTS | 1.50 | 1.22 [symbol] (−18.4%) |

[symbol]

Indicates a statistically significant (p ≦ 0.05) increase compared to Baseline [symbol]
Indicates a statistically significant (p ≦ 0.05) decrease compared to Baseline

Note that each of the significant differences are improvements compared to Baseline.

The subjective results for this anti-aging treatment parallel the clinical assessment with all subjective assessments for all parameters (smoothness, softness, lines & wrinkles, radiance, “younger-looking”, and overall efficacy) rated as significantly better at 8 weeks vs. baseline.

What is claimed is:

1. A method for beneficially treating human skin aging comprised of the following steps:
[a] producing a composition of a non-acidic formulation of copper and zinc chemically bound to a botanical pigment possessing antioxidant qualities; said composition containing a botanical copper-antioxidant-pigment complex and a botanical zinc-antioxidant-pigment complex;
[b] locating areas on the skin that show signs of photodamage, environmental damage, or aging;
[c] applying topically to said skin areas said composition of copper-botanical antioxidant complex and zinc-botanical antioxidant complex, the composition resulting in enhanced penetration of the complexes;
[d] simultaneously delivering pH-activated copper and zinc ions and plant-based antioxidant pigment to said skin damaged areas to treat said areas and to protect against further skin damage.

2. The method of claim 1 wherein said composition is a topical pharmaceutical, cosmeceutical or cosmetic formulation.

3. The method of claim 1 wherein the skin aging, photodamage or environmental damage is manifested by one or more of the following visible signs or symptoms of the skin: skin laxity or sagging, lentigenes or age spots, skin blotchiness, skin sallowness, fine lines and wrinkles, coarse wrinkles, uneven pigmentation or dyschromia, enlarged pores, dryness, and roughness.

4. The method of claim 1, wherein the skin penetration of the copper-antioxidant pigment complex and zinc-antioxidant pigment complex is enhanced by encapsulating the copper-antioxidant pigment complex and zinc-antioxidant pigment complex within a liposome shell.

5. The method of claim 1, wherein the skin penetration of the copper-antioxidant pigment complex and zinc-antioxidant pigment complex are enhanced by one or more of the skin penetrating ingredients that include pentylene glycol, isopentyl glycol, 1, 3-butylene glycol, 1,4-butylene glycol, and ethoxydiglycol, or mixtures of these diglycols.

6. The method of claim 1, wherein the copper-antioxidant pigment complex is sodium-copper-chlorophyllin.

7. The method of claim 1, wherein the zinc-antioxidant pigment complex is sodium-zinc-chlorophyllin.

8. The method of claim 1, wherein the composition is applied to any skin area of the human body showing signs of aging, photodamage, environmental damage or the like.

9. A composition for the topical treatment of human skin aging, due to photodamage or other environmentally-induced damage containing a non-acidic, aqueous, liposomal dispersion of sodium-copper-chlorophyllin and sodium-zinc-chlorophyllin in a pharmaceutical, cosmeceutical or cosmetic vehicle.

10. The composition of claim 9 wherein the sodium-copper-chlorophyllin is used at a concentration in the range of 0.005% to 0.5% by weight of the total composition.

11. The composition of claim 9 wherein the sodium-zinc-chlorophyllin is used at a concentration in the range of 0.005% to 0.5% by weight of the total composition.

12. The composition of claim 9, wherein the composition is enhanced through the addition of other topical anti-aging treatment materials that can include one or more of peptides, sunscreens, isoflavones, flavenoids, isoprenoids, quinones, carotenoids, retinoids, metalloprotease enzyme inhibitors, or other recognized anti-aging actives.

13. The method of claim 1, wherein the composition is in the form of a liquid, gel, spray, lotion, cream, or solid.
